(19) United States
(12) Patent Application Publication
(10) Pub. No.: US 2016/0174818 A1
(43) Pub. Date: Jun. 23, 2016

(54) CALIBRATED MEDICAL IMAGING DEVICES AND RELATED METHODS

(71) Applicant: Boston Scientific Scimed, Inc., Maple Grove, MN (US)

(72) Inventors: Kirsten VIERING, Watertown, MA (US); Russell P. DRESHER, Hudson, MA (US); Amy LEVASSEUR, Holliston, MA (US); James PETERSON, Yarmouth, ME (US); Paul D. AQUILINO, Walpole, MA (US)

(73) Assignee: Boston Scientific Scimed, Inc., Maple Grove, MN (US)

(21) Appl. No.: 14/973,971

(22) Filed: Dec. 18, 2015

Related U.S. Application Data
(60) Provisional application No. 62/094,720, filed on Dec. 19, 2014.

(51) Int. Cl. A61B 1/00; A61B 1/04; A61B 1/07

(52) U.S. Cl. CPC A61B 1/00057; A61B 1/00009; A61B 1/0002; A61B 1/07; A61B 1/04

(57) ABSTRACT

According to aspects of the present disclosure, a medical device may include an elongated member extending from a proximal end to a distal end, and an imaging component configured to receive image data in the body. The medical device also may include one or more memory components positioned in the medical device and configured to store image calibration data extracted from the image data received from the imaging component. In addition, the medical device may include a connector configured to connect the medical device to a control unit external to the medical device, wherein the control unit is configured to process the image calibration data stored on the one or more memory components and generate an image based on the processed image calibration data.

### CROSS-REFERENCE TO RELATED APPLICATIONS

This application claims the benefit of U.S. Provisional Application No. 62/094,720, filed Dec. 19, 2014, the disclosure of which is incorporated herein by reference in its entirety.

### TECHNICAL FIELD

Various aspects of the present disclosure relate generally to medical systems and devices. In particular, exemplary embodiments relate to endoscopic medical devices for enhanced visualization. Embodiments of the present disclosure also cover methods of manufacturing and using such systems and devices.

### BACKGROUND

Medical devices are often inserted into the body to perform a therapeutic or diagnostic procedure inside a patient's body. An example of such a device is an endoscope, which is a flexible instrument introduced into the body for diagnostic or therapeutic purposes. Typically, endoscopic devices are inserted into the body through an opening (a natural opening or an incision), and are delivered to a work site inside the body through a body channel, such as, for example, the esophagus. Imaging devices incorporated in endoscopes allow a surgeon to see the work site from outside the body and remotely operate the endoscope to perform a desired diagnostic/therapeutic procedure at the work site. There are many different types of endoscopes in use today and embodiments of the current disclosure may be applied with any of these different types of endoscopes or other medical devices. In general, embodiments of the current disclosure may be applicable with any type of medical device that can be inserted into a body, and that allows a surgeon outside the body to visualize a region inside the body. For the sake of brevity, however, the novel aspects of the current disclosure will be described with reference to an endoscope.

In a typical application, a distal end of an endoscope may be inserted into the body through an opening in the body. This opening may be a natural anatomic opening, such as, for example, the mouth, rectum, vagina, etc., or an incision made on the body. The endoscope may be pushed into the body such that the distal end of the endoscope proceeds from the point of insertion to a region of interest (work site) within the body by, e.g., traversing a body channel or cavity. The endoscope may include one or more lumens extending longitudinally from the proximal end to the distal end of the endoscope. These lumens may deliver various diagnostic/treatment devices from outside the body to the work site to assist in the performance of the intended procedure at the work site.

Among others, these lumens may include an imaging lumen that may include an imaging component to capture images of the work site and deliver the image outside the body for viewing. Endoscopic visualization is used to diagnose and/or treat any number of conditions in the gastric, pulmonary, and urologic tracts. Endoscopes are required to not only navigate to the target site, but also to provide adequate visualization for diagnosis and/or treatment. Adequate visualization requires sufficient image quality to represent details within the anatomy under observation.

Often, endoscopes use high-end video cameras that produce superior video quality. Often, this is achieved through a large array of pixels and robust supporting electronics. These aspects reduce camera-to-camera and pixel-to-pixel variability such that the same high quality video is achieved for all endoscopes. This benefit comes at the cost of camera size (e.g., more pixels and robust supporting electronics require a large area) and component pricing (e.g., high price). Endoscopes designed to visualize small diameter anatomy may not be able to afford the size increases to support large pixel arrays or supporting electronics. In these cases, size and image quality compete. Furthermore, disposable endoscopes offer design constraints between cost and image quality. Reducing size and cost of the camera often results in lower image quality due to noise and/or less available information.

Therefore, improved devices and methods are needed for maintaining a compact design of the endoscope while maintaining high quality imaging.

### SUMMARY

Aspects of the present disclosure relate to, among other things, medical devices and methods of imaging portions of the body.

According to aspects of the present disclosure, a medical device may include an elongated member extending from a proximal end to a distal end, an imaging component configured to receive image data in the body, one or more memory components positioned in the medical device and configured to store image calibration data extracted from the image data received from the imaging component, and a connector configured to connect the medical device to a control unit external to the medical device, wherein the control unit is configured to process the image calibration data stored on the one or more memory components and generate an image based on the processed image calibration data.

In addition or alternatively, the device may include one or more of the following features. The stored image calibration data may include defective pixel data and/or pixel dimension data. The one or more memory components may store device identifying data. The imaging component may be positioned on the distal end of the elongated member. The elongated member may include multiple lumens and the imaging component may be disposed in one of the multiple lumens. The medical device also may include a handle having the connector and the handle may be configured to move the elongated member through an opening in the body to a worksite.

According to aspects of the present disclosure, a computer implemented method of controlling medical device image quality, using a computer system, may include: illuminating the imaging component with low angle light; receiving light scattering data from an imaging component of a medical device disposed in the enclosure illuminated with the low angle light; determining, based on the light scattering data, if the imaging component of the medical device is free of debris; illuminating the imaging component of the medical device with even illumination from an illumination source of the medical device, the even illumination having a pre-determined illumination value; receiving image data from the imaging component of the medical device responsive to the even illumination; varying the pre-determined illumination value from the illumination source of the medical device and updating the image data based on updated imaging data from the imaging component of the medical device responsive to the varied pre-determined illumination value; generating image calibration data for the medical device based on the updated image data; and recording the image calibration data for the medical device in a memory of the medical device.

In addition or alternatively, the method may include one or more of the following features. Recording in the memory of the medical device, identifying information of the medical device. Determining light leakage data from the medical device comprising. Receiving, from the imaging component of the medical device, image data responsive to light transmitted through optical fibers of the medical device from a light source. Processing the image data responsive to the light transmitted through the optical fibers of the medical device from the light source to determine light leakage from the medical device. The step of processing the image data to determine light leakage from the medical device comprises executing one or more image processing algorithms. The step of generating image calibration data may include processing a pixel response based on each individual's camera response to the illumination. The step of varying the pre-determined illumination value may include varying an intensity of the illumination. A step of processing the image calibration data to generate an image based on the processed calibration data. The step of processing the calibration data may include determining one or more image processing algorithms to apply to the image data. Recording in the memory of the medical device, identifying information of the medical device and determining compatibility of the medical device with a medical system based on the identifying information of the medical device. The low-angle light is generated by a ring light.

According to aspects of the present disclosure, a system of controlling medical device image quality may include: a data storage device storing instructions for controlling medical device image quality; and a processor configured to execute instructions to perform a method including: illuminating an enclosure with low angle light; receiving image data from an imaging component of a medical device disposed in the enclosure illuminated with the low angle light; determining, based on the image data, if the imaging component of the medical device is free of debris; illuminating the imaging component of the medical device with even illumination from an illumination source of the medical device, the even illumination having an illumination value; receiving image data from the imaging component of the medical device responsive to the even illumination; varying the even illumination value from the illumination source of the medical device and updating the image data based on updated imaging data from the imaging component of the medical device responsive to the varied even illumination value; generating image calibration data for the medical device based on the updated image data; and recording the image calibration data for the medical device in a memory of the medical device.

According to aspects of the present disclosure, a system may include a control unit, and a medical device, the medical device including: an elongated member extending from a proximal end to a distal end; an imaging component configured to receive image data in the body; one or more memory components positioned in the medical device storing image calibration data from the image data received from the imaging component; and a connector configured to connect the medical device to the control unit external to the medical device, wherein the control unit may be configured to process the image calibration data stored on the one or more memory components and generate an image based on the processed image calibration data.

According to aspects of the present disclosure, a method of controlling medical device image quality, using a computer system, may include: providing an enclosure; illuminating the enclosure with low angle light; providing a medical device comprising an imaging component, and disposing the medical device in the enclosure; receiving light scattering data from the imaging component of the medical device disposed in the enclosure illuminated with the low angle light; determining, based on the light scattering data, if the imaging component of the medical device is free of debris; illuminating the imaging component of the medical device with even illumination from an illumination source of the medical device, the even illumination having a pre-determined illumination value; receiving image data from the imaging component of the medical device responsive to the even illumination; varying the pre-determined illumination value from the illumination source of the medical device and updating the image data based on updated imaging data from the imaging component of the medical device responsive to the varied pre-determined illumination value; generating image calibration data for the medical device based on the updated image data; and recording the image calibration data for the medical device in a memory of the medical device.

It may be understood that both the foregoing general description and the following detailed description are exemplary and explanatory only and are not restrictive of the claimed features.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a schematic view of an embodiment of a distal end of a medical device transmitting image quality data.

FIG. 2 is another view of the distal end of the medical device of FIG. 1 transmitting image quality data in response to illumination from low angle illumination source.

FIG. 3 is a block diagram of an exemplary method for controlling medical device image quality, according to an exemplary embodiment of the present disclosure.

FIG. 4 is a block diagram of an exemplary method for processing image calibration data for a medical device, according to an exemplary embodiment of the present disclosure.

FIG. 5 is an illustration of a graph showing s value as a function pixel value.

### DETAILED DESCRIPTION

Overview

The present disclosure is drawn to medical devices and methods of using medical devices.

Portions of the medical device may be used to capture images of a patient's body during a medical procedure. The medical device also may include components to store data about the imaging properties of the medical device. In addition or alternatively, the medical device may include the same or different components to store identifying information about the medical device. The medical device may be connected to an external device, which may retrieve the data stored on the medical device and process the data to control the display of the images captured by the medical device to improve the quality of the images.

Reference will now be made in detail to aspects of the present disclosure, examples of which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject.

Exemplary Aspects

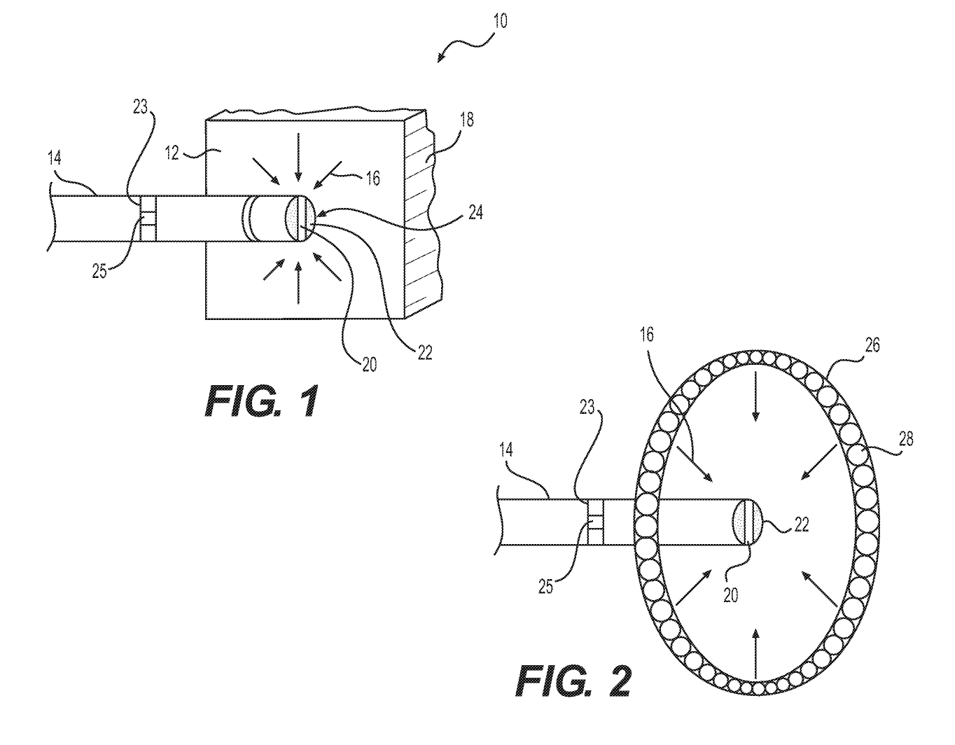

FIG. 1 illustrates an illumination apparatus 10 for illuminating a medical device 14 with low-angle light 16. The apparatus 10 may be configured for detection of any debris on the distal end 24 of the medical device 14 and/or any leakage of light from the medical device 14. The medical device 14 may be any suitable medical device for insertion in the body (e.g. an endoscope), and may include one or more internal lumens for insertion of other medical/diagnostic tools, therapeutic matter, and/or removal of matter. The one or more lumens may have an opening at the distal tip of the medical device 14. The medical device 14 may include one or more internal illumination sources 20, and one or more imaging components 22, such as a camera. The illumination sources 20 and/or imaging components 22 may be positioned at any suitable location of the medical device 24, for example at the distal end 24 of the medical device 14. The imaging component 22 may include, for example, an image sensor which may include a CMOS imaging sensor chip or other solid state image sensor such as a low light sensitive, low noise, CMOS color imager with a possible range of image resolution determined on the requirements as well as cost and dimensional restraints. Video output of the imaging component 22 may be according to a custom signal scheme, a conventional digital or analog format, including PAL or NTSC or high definition video format. Following image processing, the video format may follow a standard video signal to provide compatibility with suitable visualization components (e.g. monitors) and/or recording equipment.

The medical device 14 also may include a circuit board 23 having various components, such as one or more memory chips 25 and/or one or more processors configured to process the image data (e.g. calibration data) stored on the memory chip 25 and generate image processing parameters. Alternatively, the processor may be in an external processing unit. The image processing parameters generated by the processor on the circuit board 23 may be saved on the memory chip 25. The circuit board 23 may be positioned at any suitable location of the medical device 14, for example, within the connector or handle of the medical device 14 or at the distal end 24 of the medical device 14, e.g. in an elongated shaft of an endoscope. The circuit board 23 may be configured to communicate with an external controller for retrieving data stored on the memory chip 25 in any suitable manner (e.g. via wired or wireless communication).

The apparatus 10 may be configured to facilitate detection of any debris, condensation, or other undesirable matter on the distal end 24 of the medical device 14 indicating the imaging component may not be clean. An example of facilitating the detection of an imaging component 22 that may not be clean may include determining any light detected on images generated by the imaging component 22 in response to low-angle light 16 being scattered by debris and directed into the imaging component 22 in the closed enclosure 12. Such light scattering may be indicative of the distal end 24 of the medical device 14 not being clean.

In addition, or alternatively, the apparatus 10 may be configured to detect any light leakage from the illumination source(s) 20 of the medical device 14. As shown in FIG. 1, the distal end of 24 of the medical device may be positioned in the enclosure 12. The enclosure 12 may be configured to reduce or prevent ambient light from entering, for example, by being manufactured using dark, light-absorbing materials 18. Additionally, the enclosure 12 may absorb light being emitted from the illumination source 20 of the medical device 14. The enclosure 12 may have any suitable size and shape to allow insertion of the distal end 24 of the medical device 14, reduce or prevent any ambient light from entering the enclosure 12, and allow low-angle light source 16 to illuminate the enclosure 12. The one or more illumination sources 20 of the medical device may be any suitable illumination sources, such as a light emitting diodes (LEDs) configured to provide light to the target location such that the imaging component 22 of the medical device 14 may visualize the target location. The illumination sources 20 may be internal or external to the medical device 14. The light may be transmitted to the distal end of the medical device 14 in any suitable manner, such as via optical fibers.

FIG. 2 shows an example of low-angle light 16 that may be provided in the enclosure 12 shown in FIG. 1. The low-angle light 16 may be generated by any suitable low-angle light emitting source, such as a ring light 26 having light sources 28. The light sources 28 may be any suitable light sources, such as LEDs. The low-angle light 16 may be configured to illuminate the distal end 24 of the medical device 14 at various low-angles to provide detection of any light scattering from any debris and/or residue on the distal end 24 of the medical device 14. As described in further detail below, light scattering or light leakage may reduce the accuracy of any imaging calibration data from the imaging component 22 of the medical device 14. As further described below, the image data generated from the imaging component 22 may be electronically stored in memory as calibration data, which may be processed to improve the image quality of the medical device 14.

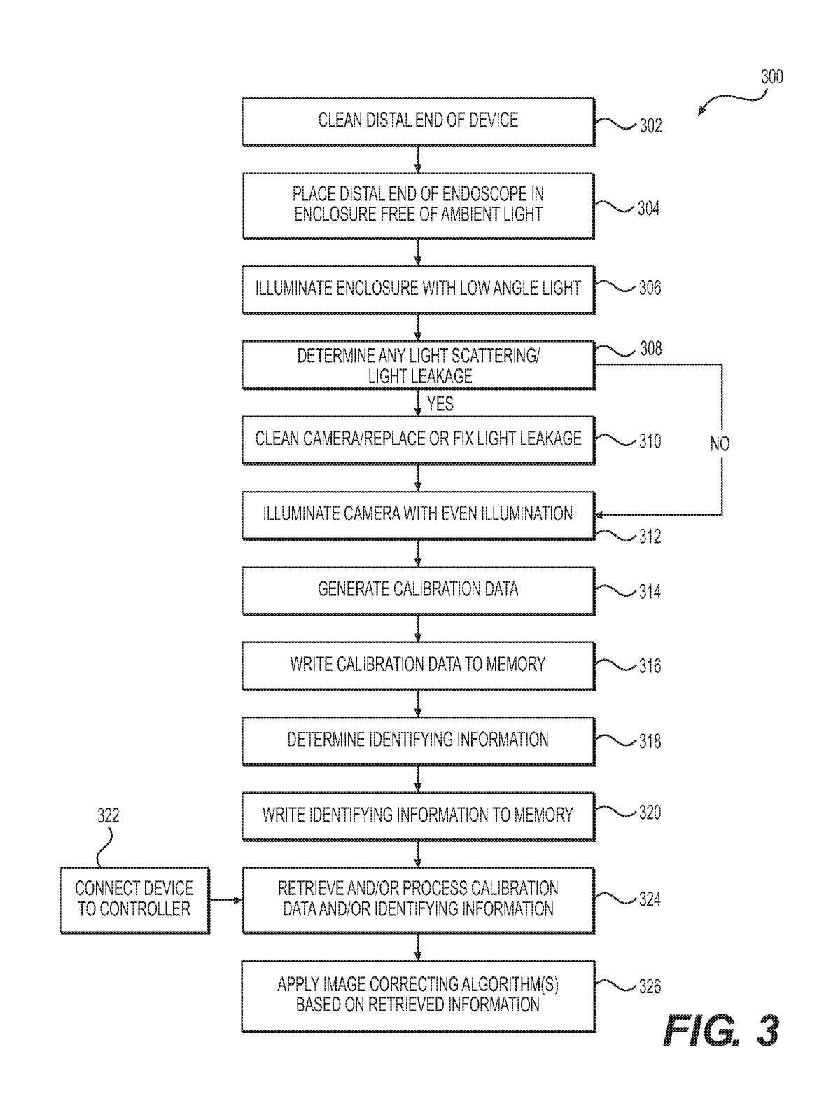

FIG. 3 shows an example of a method 300, which may be used to improve the quality of images generated by a medical device, such as the imaging component 22 of medical device 14. Some of the acts of the method 300 may be conducted during the manufacture (e.g. in-line manufacture) of the medical device 14. Execution of 302 through 320 may simplify the use of the medical device 14 by the user. The method 300 may include 302, corresponding to cleaning of the distal end 24 of the medical device 14 in any suitable manner. In one example of 302, the lens of the imaging component 22 of the medical device 14 may be cleaned with pressurized air, water, and/or a non-abrasive cleaner to remove any residue and/or debris, which may interfere with calibrating the imaging component 22.

The distal end 24 of the medical device 14 having the imaging component 22 may then be placed in an enclosure, such as enclosure 12, at 304. As discussed above, the enclosure 12 may be configured to reduce or prevent any ambient light from entering the enclosure 12. 306 may include providing low-angle light 16 around the distal end 24 of the medical device 14. 306 also may include the imaging component 22 capturing images while being illuminated with the low-angle light 16. The low-angle light 16 may allow detection of any residue or debris on the distal end 24 of the medical device 14 by detecting any light scattered by the debris. For example, any debris on the imaging component 22 (e.g. the lens of a camera) at the time of generating image calibration data may lead to incorrect image calibration data.

While the distal end 24 of the medical device 14 is inside the enclosure 12, as described in 304, the imaging component 22 may be illuminated with the low-angle light 16, for example from the ring light 26 shown in FIG. 2. This low-angle light 16 may not be visible in the imaging component 22 image, as this low-angle light 16 may not pass through the aperture of the imaging component 22. The low-angle light 16 instead may be absorbed by the dark material 18 within the enclosure 12 such that light may not be scattered, which could, in turn, be visible in the image captured by the imaging component 22.

However, any debris or residue that may be on a distal surface of the lens of the imaging component 22 may cause light scattering at the front of the imaging component 22. This scattered light may be collected by the lens of the imaging component 22 and pass through the aperture. At 308, any scattered light caused by debris on the lens of the imaging component 22 may be determined as any such scattered light may be visible in the image captured by the imaging component 22 of the medical device 14. Either an operator and/or pattern recognition software may be used to differentiate between imaging components free of debris and those that may require further cleaning. The operation software may be stored on and/or processed in memory in the medical device 14 or on an external device electronically connectable to the medical device 14.

In addition to, or alternatively, 308 may include determining light leakage from the medical device 14. A same or similar configuration as described above in reference to determining any light scattering may be used to determine any light leakage from the medical device 14. The light leakage may be due to light from the illumination source 20 leaking on its passage from the illumination source 20 and out of the distal end 24 of the medical device 14. Such light leakage may be from any illumination fibers used for transmitting the light from the illumination source 20 and out of the distal end 24 of the medical device 14. To determine such light leakage, the low-angle light 16 may be turned off. Illumination may then be provided by an illuminating source 20 of the medical device and/or an external light source that may be connected to a proximal end of the illumination fibers. Any light emitted from the distal end of the fibers may be absorbed by the enclosure 12 such that any reflected light may not be visible in images captured by the imaging device 22. However, if light leakage is present, it may be detected on the images captured by the imaging component 22. Either an operator and/or pattern recognition software may be used to differentiate between medical devices free of light leakage and those that exhibit light leakage. For example, for determining light leakage, the detection algorithm may identify any changes in the dark level pixel values in a localized region. To determine this, two images may be taken: an image without light emitted from the fiber(s), and an image with light emitted from the fiber(s). Once these image are obtained, the difference in pixel values for the image may be calculated. The result may be a new ‘image’ that may show the difference in each pixels' value between the image without light emitted from the fibers and the image with light emitted from the fibers. The algorithm may then select a local area, for example an area having a size of 10×10 pixels, and determine if the average pixel value in that area has varied by more than a predetermined value. If the average pixel value has varied by more than the predetermined value, then light leakage may have been detected. If the average pixel value has not varied by more than the predetermined value, then no light leakage may be present. The pixel region may then be moved over by 1 pixel in one dimension and the analysis may be repeated. Thus, using the same method, a complete image size may be analyzed.

Any light scattering indicative of debris and/or any light leakage may indicate a need for 310, corresponding to cleaning and/or replacing or fixing the medical device to correct any interference due to debris or light leakage which may interfere with generating accurate image calibration data which may be used to reduce the fixed pattern noise of the imaging component 22.

In addition, in order to determine whether the imaging component 22 of the medical device provides adequate visualization, the illumination source 20 and, if applicable, the optical fibers may be checked and the light measured to determine if it is transmitting sufficient amounts of light to the distal end 24 of the medical device 14. A light output check may therefore also be included in the method 300. Depending on the application and type of illumination, different measurement devices may be used to measure the amount of light emitted by the illumination source 20. Such measurement devices may include, for example, photo detectors, integrating spheres, and/or spectrometers.

At 312, the imaging component 22 may be illuminated with even illumination to obtain various properties of the imaging component, such as pixel properties. An example, of a method of obtaining pixel properties may include assessing each image pixel's response relative to its neighbors' responses at varying illumination levels. This even illumination of the imaging component 22 at varying illumination levels may be provided, for example, by using a backlight, and/or by using an integrating sphere, where even illumination may be achieved by numerous reflections of the light. The size of the backlight may exceed the field of view of the imaging component 22 to provide all pixels with the same or similar amount of light. The illumination level of the backlight may be varied (quasi-) continuously such that the imaging component 22 may detect a minimum amount or no light and may achieve maximum saturation. Similar illumination requirements may be applied to the integrating sphere. The reflectivity of the sphere may be sufficient to provide an even illumination profile. To gather the image data at varying levels of illumination, the even illumination intensity may be varied, such that image data from the imaging component 22 may be gathered at a number of different image saturation levels, from a dark signal (e.g., no light impinging upon the camera) to 100% image saturation. This image data then may be used to determine each pixel's response curve and generate the image calibration data for the image component 22 of the medical device at 314.

Correction values based on the response curve data for each pixel, or alternatively, the response curve data for each pixel, may be saved in digital memory at 316 as retrievable image calibration data. The image calibration data may be processed, either by the processor in the medical device 14, or a processor external to the medical device 14, to generate image processing parameters and/or optimize the image generated by the medical device 14. Various characteristics of the image captured by the imaging component 22 in response to the varied illumination may be saved as image calibration data on the memory chip 25, such as the frame width and height in pixels of the image if the resolution of the imaging component 22 is unknown, the identification, and a listing of any defective pixels, as well as characteristics of each pixel in the image. The circuit board 23 may be located in any suitable portion of the medical device, for example, a handle portion, connector, and/or distal portion 24 of the medical device 14.

In steps 318 and 320, the same or a different interface used to write the image calibration data on the memory chip 25 also may be used to identify and write any additional suitable information, such as the product identifier, serial/LOT number, manufacturing date and site, and a hardware/software version identifier of the medical device 14.

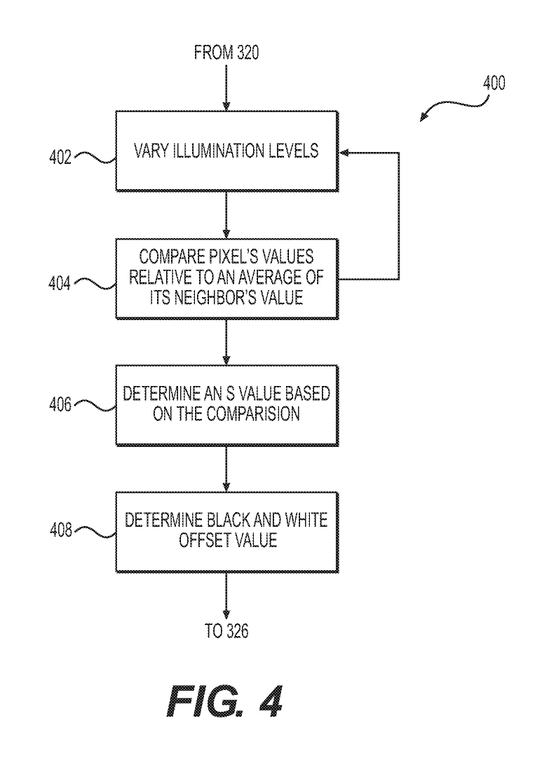

The method 300 also may include 322, corresponding to connecting the medical device 14 to an external controller having one or more processors. 322 may be performed during the method 300 and/or separately from method 300 to confirm that calibration has been correctly performed. The processor(s) of the external controller may be configured to retrieve and/or process the image calibration data stored in memory of the medical device 14 as part of 324. The medical device 14 may be connected to the external controller either via wires or wirelessly, in any suitable manner. The external controller may include one or more software programs configured to process the retrieved image calibration data and optimize the images. For example, one method of processing the image calibration data is shown in FIG. 4.

Referring now to FIG. 4, method 400 of processing image calibration data may include 402, corresponding to varying even illumination levels to gather the image data at varying levels of illumination, as described above. The even illumination intensity may be varied, such that image data from the imaging component 22 may be gathered at any suitable number of different image saturation levels, from a dark signal (e.g., no light impinging upon the camera) to 100% image saturation.

At 404, a value from each pixel in the image generated by the imaging component at each illumination intensity level may be compared to values of a statistical value of the pixel's neighboring pixel(s). For example, the value for each pixel may be compared relative to an average (mean or median) of its neighboring pixel(s). At 406, a value “s” may be assigned, such that when the pixel value and the single s value are added, they may match or substantially match the average value of the neighboring pixels.

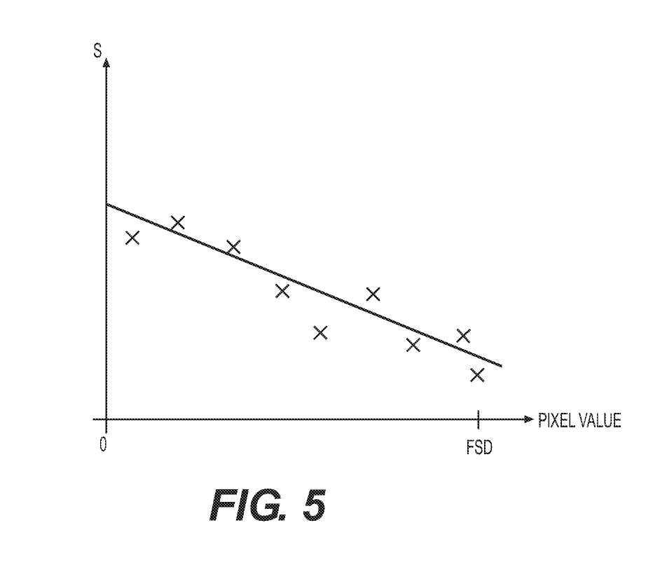

At 408, at least one black and white offset value may be determined. FIG. 5 illustrates an example of the value S varying with the pixel's value (FSD=full scale digital=maximum pixel value), as indicated by points X in FIG. 5. A linear (least squares) fit (sloped line in FIG. 5) may then be used to determine the ideal black and white offset value to minimize the error between the pixel's value and the average of its neighbor's. The black and white offset values may correspond to the values of the least squares fit at the points 0 and FSD, e.g. the two points (0, black offset level) and (FSD, white offset level) and/or at any other appropriate point sufficient to reconstruct the least squares fit line. The black and white offset values may be used in the image correcting algorithm(s) of 326. It is contemplated that method 400 may be one of multiple methods performed as part of 324 for processing image calibration data.

For example, the external controller may process the defective pixel list stored as the image calibration data to substitute any defective pixels with neighboring pixels. This may effectively remove defective pixels on the processed image presented to the user. Storing the image calibration data on the memory of the medical device 14 (at, for example, 320 in FIG. 3) may allow the external controller to read the image calibration data and to apply a suitable image correcting algorithm (at, for example, 326 in FIG. 3).

In some examples, the image correcting algorithm may be selected and applied based on processing the retrieved calibration data by the external controller. Alternatively, or additionally, the retrieved data may be processed by a processor in the medical device (e.g. in the circuit board 23) and stored in the memory 25. The suitable image correcting algorithm may include any suitable data manipulation, for example, the algorithm may correct offset values at 326. Thus, the method 300 may reduce/remove fixed pattern noise in the imaging component 322.

In addition, the external controller may retrieve and process any device identifying data previously stored in the memory of the medical device at 320. Processing of the device identifying information stored in the memory of the medical device may allow for discrimination between various medical devices 14. For example, processing the identifying data of the device may identify compatible and non-compatible medical devices manufactured, for example, by a different company. This device identifying data may mitigate safety risks such as using incompatible medical devices.

In another example, the device identifying data stored in the memory of the medical device at 320 also may be retrieved and processed by the external controller to apply specific imaging settings. For example, an external controller may determine, based on the device identifying data, which device may be used in different parts of the anatomy. Different anatomies may benefit from different image processing algorithms. In this manner, the device identifying data may be processed by the external controller to determine which image processing software and/or which particular kind of algorithm(s) to apply. Thus, further optimization may be achieved by selectively choosing the appropriate image processing algorithm based on device type and the likely anatomy to be imaged by the devices of this type.

One of the external controllers discussed above may include or be connected to a general purpose computer hardware platform and also may be connected to a network or host computer platform (not shown) as may typically be used to implement a server executing illumination and/or visualization as described above. It is believed that those skilled in the art are familiar with the structure, programming, and general operation of such computer equipment should be self-explanatory.

A platform for a server or the like may include a data communication interface for packet data communication. The platform may also include a central processing unit (CPU) in the form of one or more processors, for executing program instructions. The platform typically includes an internal communication bus program storage and data storage for various data files to be processed and/or communicated by the platform such as ROM and RAM, although the server often receives programming and data via network communications. The hardware elements, operating systems, and programming languages of such equipment are conventional in nature, and it is presumed that those skilled in the art are adequately familiar therewith. The server also may include input and output ports to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The disclosed medical devices and methods may be utilized in any suitable application involving illumination and/or visualization in the body during a therapeutic and/or diagnostic medical procedure. Any aspect set forth herein may be used with any other aspect set forth herein. The devices may be used in any suitable medical procedure, and may be advanced through any suitable body lumen and body cavity. For example, the devices described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed medical devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
an elongated member extending from a proximal end to a distal end;
an imaging component configured to receive image data in the body;
one or more memory components positioned in the medical device and configured to store image calibration data extracted from the image data received from the imaging component; and
a connector configured to connect the medical device to a control unit external to the medical device, wherein the control unit is configured to process the image calibration data stored on the one or more memory components and generate an image based on the processed calibration data.

2. The medical device of claim 1, wherein the stored image calibration data comprises defective pixel data.

3. The medical device of claim 1, wherein the stored image calibration data comprises pixel dimension data.

4. The medical device of claim 3, wherein the one or more memory components further stores device identifying data.

5. The medical device of claim 1, wherein the imaging component is positioned on the distal end of the elongated member.

6. The medical device of claim 1, wherein the elongated member comprises multiple lumens and the imaging component is disposed in one of the multiple lumens.

7. The medical device of claim 1, further comprising a handle comprising the connector and configured to move the elongated member through an opening in the body to a worksite.

8. A computer implemented method of controlling medical device image quality, using a computer system, the method comprising:
illuminating an enclosure with low angle light;
receiving light scattering data from an imaging component of a medical device disposed in the enclosure illuminated with the low angle light;
determining, based on the light scattering data, if the imaging component of the medical device is free of debris;
illuminating the imaging component of the medical device with even illumination from an illumination source of the medical device, the even illumination having a pre-determined illumination value;
receiving image data from the imaging component of the medical device responsive to the even illumination;
varying the pre-determined illumination value from the illumination source of the medical device and updating the image data based on updated imaging data from the imaging component of the medical device responsive to the varied pre-determined illumination value;
generating image calibration data for the medical device based on the updated image data; and
recording the image calibration data for the medical device in a memory of the medical device.

9. The method of claim 8, further comprising, recording in the memory of the medical device, identifying information of the medical device.

10. The method of claim 9, wherein the identifying information comprises manufacturing information of the medical device, and software information of the medical device.

11. The method of claim 8, further comprising, determining light leakage data from the medical device comprising:
receiving, from the imaging component of the medical device, image data responsive to light transmitted through optical fibers of the medical device from a light source; and
processing the image data responsive to the light transmitted through the optical fibers of the medical device from the light source to determine light leakage from the medical device.

12. The method of claim 11, wherein the step of processing the image data to determine light leakage from the medical device comprises executing one or more image processing algorithms.

13. The method of claim 11, wherein the step of generating image calibration data comprises processing a pixel response based on the varied pre-determined illumination values.

14. The method of claim 8, wherein the step of varying the pre-determined illumination value comprises varying an intensity of the illumination.

15. The method of claim 8, further comprising a step of processing the image calibration data to displaying an image from the imaging component of the medical device based on the processing of the image calibration data.

16. The method of claim 15, wherein the step of processing the image calibration data comprises, determining one of more image processing algorithms to apply to the image data.

17. The method of claim 8, further comprising recording in the memory of the medical device, identifying information of the medical device and determining compatibility of the medical device with a medical system based on the identifying information of the medical device.

18. The method of claim 8, wherein the low-angle light is generated by a ring light.

19. A system of controlling medical device image quality, the system comprising:
a data storage device storing instructions for controlling medical device image quality; and
a processor configured to execute instructions to perform a method including:
illuminating an enclosure with low angle light;
receiving light scattering data from an imaging component of a medical device disposed in the enclosure illuminated with the low angle light;
determining, based on the light scattering data, if the imaging component of the medical device is free of debris;
illuminating the imaging component of the medical device with even illumination from an illumination source of the medical device, the even illumination having a pre-determined illumination value;
receiving image data from the imaging component of the medical device responsive to the even illumination;
varying the pre-determined illumination value from the illumination source of the medical device and updating the image data based on updated imaging data from the imaging component of the medical device responsive to the varied pre-determined illumination value;
generating image calibration data for the medical device based on the updated image data; and
recording the image calibration data for the medical device in a memory of the medical device.
